(19) United States
(12) Patent Application Publication
(10) Pub. No.: US 2011/0166384 A1
(43) Pub. Date: Jul. 7, 2011

(54) CATALYST FOR PRODUCING ACRYLIC ACID AND PROCESS FOR PRODUCING ACRYLIC ACID USING THE CATALYST

(76) Inventors: Michio Tanimoto, Himeji-shi (JP); Nobuyuki Hakozaki, Himeji-shi (JP)

(21) Appl. No.: 12/999,789

(22) PCT Filed: Sep. 25, 2009
(86) PCT No.: PCT/JP2009/066655

§ 371 Date: Dec. 17, 2010

(30) Foreign Application Priority Data
Sep. 30, 2008 (JP) 2008-252081

(51) Int. Cl. C07C 51/235; B01J 23/28; C07C 51/215

(52) U.S. Cl. 562/535; 502/312; 562/549

(57) ABSTRACT

A catalyst for producing acrylic acid from propane and/or acrolein by gas-phase catalytic oxidation with molecular oxygen-containing gas, comprising a catalytic active component containing molybdenum and vanadium as essential elements, and having a crystallinity T of 5% or more and 20% or less in the 20 range of 5° to 90°, measured by an X-ray diffraction analysis with Cu-Kα radiation; and a process for producing acrylic acid by gas-phase catalytic oxidation of propane and/or acrolein with molecular oxygen, comprising the step of conducting the gas-phase catalytic oxidation in the presence of the catalyst. According to the catalyst and the process for producing acrylic acid of the present invention, it is possible to stably produce acrylic acid at a high yield for a long period.

### TECHNICAL FIELD

The present invention relates to a catalyst suitable for producing acrylic acid from propane and/or acrolein by gas-phase catalytic oxidation with molecular oxygen and a process for producing acrylic acid using the catalyst.

### BACKGROUND ART

Acrylic acid is industrially important as a raw material for various synthetic resins, paints and plasticizers, and recently, its importance as a raw material for an absorbent polymer has been increasing. The most common process for producing acrylic acid is a two-stage oxidation process in which acrolein is produced from propylene by gas-phase catalytic oxidation and then acrylic acid is produced from the obtained acrolein by gas-phase catalytic oxidation.

Meanwhile, in view of price difference between propane and propylene, a process for producing acrylic acid by one-stage oxidization of propane has been developed recently and various proposals have been made. As a catalyst for producing acrylic acid by gas-phase catalytic oxidation of propane and/or acrolein with molecular oxygen-containing gas, a molybdenum-vanadium-containing catalyst has been mainly investigated, and various modifications have been made by many companies for the purpose of improving catalyst performance.

As for producing acrylic acid from acrolein by gas-phase oxidation, there have been proposed various catalysts which have high activity and provides a high yield of acrylic acid and are usable even under high load conditions. For example, Patent literature 1 discloses a catalyst having a peak intensity ratio between a peak attributed to VMo₃O₁₁ and a peak attributed to V₂O₅ in a specific range, measured by an X-ray diffraction analysis with Cu-Kα radiation. Patent literature 2 discloses a catalyst having a specific maximum peak measured by an X-ray diffraction analysis with Cu-Kα radiation. Patent literature 3 discloses a catalyst having a specific peak in the 28 range of 5° to 50°, measured by an X-ray diffraction analysis with Cu-Kα, radiation. Further, Patent literature 4 discloses, as a catalyst for obtaining acrylic acid by gas-phase oxidation of propane, a catalyst having a peak with the maximum intensity at a specific reflection angle and a catalyst that does not have a peak at a specific reflection angle by an X-ray diffraction analysis.

### CITATION LIST

### Patent Literature

### Patent Literature 1

Japanese Unexamined Patent Application Publication No. 2002-233757

### Patent Literature 2

Japanese Unexamined Patent Application Publication No. 8-299797

### Patent Literature 3

Japanese Unexamined Patent Application Publication No. 9-194213

### Patent Literature 4

Japanese Unexamined Patent Application Publication No. 2004-504288

### SUMMARY OF INVENTION

### Technical Problem

Acrylic acid is produced as much as several millions of tons per year worldwide, and the demand therefor as a raw material for an absorbent polymer is still increasing. In consideration of recent jumps in the raw material prices, enhancing the yield of acrylic acid in an industrial scale by just 0.1% would have a significant meaning in terms of economy. Although the catalysts disclosed in Patent literatures 1 to 4 are somewhat improved in the intended catalyst performance such as the yield of acrylic acid, catalyst life and the like, there is still room for improvement in an industrial scale.

In the catalyst disclosed in Patent literature 1, the yield of acrylic acid in an initial stage of the reaction is relatively high. However, after continuous 8000 hours of the reaction, the yield of acrylic acid, which was 94.4 mol % in the initial stage of the reaction, decreases to 92.9 mol %, even under a condition of a low raw material acrolein concentration of 4.5%. The catalyst of Patent literature 1 is therefore still insufficient from the viewpoint of the catalyst life. Patent literature 2 indeed discloses a high yield of acrylic acid, it is merely a laboratory-scale evaluation result obtained by using only 30 mL of a catalyst. In addition, in Patent literature 2, the catalyst life is not evaluated. In Patent literature 3, it is disclosed of relatively high acrylic acid selectivity in an initial stage of the reaction; however, similarly to Patent literature 1, it is evaluated under a condition of a low raw material acrolein concentration of 5.0% and merely in the initial stage of the reaction, and further, the catalyst life is not evaluated. There is unknown about the catalyst performance when acrylic acid is continuously produced by the reaction over a long period. In Patent literature 4, it is disclosed of a relatively high yield of acrylic acid in a process using gas-phase oxidation of propane; however, the performance is evaluated merely in an initial stage of the reaction, and the catalyst life is not evaluated. There is unknown about the catalyst performance when the reaction is continued over a long period. Furthermore, none of Patent literatures 1 to 4 discloses a technical idea of enhancing the yield of acrylic acid over a long period by controlling a crystallinity of a catalytic active component within an appropriate range.

The present invention has been achieved in view of the above circumstances, and an object of the present invention is to provide a catalyst which is excellent in catalyst performance such as catalytic activity and selectivity as well as in catalyst life and provides stable performance over a long period in producing acrylic acid from propane and/or acrolein.

### Solution to Problem

The catalyst of the present invention which solves the above problems is a catalyst for producing acrylic acid from propane and/or acrolein by gas-phase catalytic oxidation with molecular oxygen-containing gas, comprising: a catalytic active component containing molybdenum and vanadium as essential elements, and having a crystallinity T of 5% or more and 20% or less in the 20 range of 5° to 90°, measured by an X-ray diffraction analysis with Cu-Kα radiation. According to the catalyst of the present invention, since the crystallinity T is within the aforementioned range, the catalyst is excellent in catalytic performance such as catalytic activity and selectivity as well as in catalyst life, and provides stable performance over a long period.

In the catalyst of the present invention, it is preferred that a crystallinity ratio R, which is calculated as a ratio of a crystallinity M of the peak at 2θ=22.2±0.5° in the catalytic active component, measured by the X-ray diffraction analysis with Cu-Kα radiation, to the crystallinity T, as expressed by the following formula (1), is preferably in the range of 0.06 or more and 0.30 or less. When the crystallinity ratio R is adjusted in that range in addition to the crystallinity T, it becomes easy to obtain a catalyst for producing acrylic acid which is more excellent in catalyst performance and catalyst life and provides stable performance over a long period.

R=M/T (1)

In addition, the process for producing acrylic acid of the present invention is a process for producing acrylic acid by gas-phase catalytic oxidation of propane and/or acrolein with molecular oxygen-containing gas, comprising the step of conducting the gas-phase catalytic oxidation in the presence of the catalyst of the present invention. According to the process for producing acrylic acid of the present invention, it is possible to stably produce acrylic acid at a high yield for a long period.

### Advantageous Effects of Invention

According to the catalyst and the process for producing acrylic acid of the present invention, it is possible to stably produce acrylic acid at a high yield for a long period in producing acrylic acid by gas-phase catalytic oxidation of propane and/or acrolein with molecular oxygen.

### DESCRIPTION OF EMBODIMENTS

The catalyst for producing acrylic acid of the present invention and the process for producing acrylic acid of the present invention using the catalyst will hereinafter be explained in detail; however, the present invention is not limited to the following description, and can be put into practice after appropriate modification or variations within a range meeting the gist of the present invention in addition to the following embodiments.

The catalyst for producing acrylic acid of the present invention is a catalyst for producing acrylic acid by gas-phase catalytic oxidation of propane and/or acrolein with molecular oxygen-containing gas. A raw material gas for producing acrylic acid contains at least propane and/or propylene in addition to molecular oxygen-containing gas. The molecular oxygen-containing gas is not particularly limited as long as it contains molecular oxygen, and for example, gas consisting of only molecular oxygen may be used, or air may be used, as the molecular oxygen-containing gas.

The catalyst of the present invention comprises a catalytic active component containing molybdenum and vanadium as essential elements, and the catalytic active component has a crystallinity T of 5% or more and 20% or less in the 20 range of 5° to 90°, measured by an X-ray diffraction analysis with Cu-Kα radiation. The crystallinity T is preferably 7% or more and 15% or less. In the catalyst of the present invention, since the catalytic active component has the crystallinity T of 5% or more and 20% or less in the 20 range of 5° to 90°, measured by an X-ray diffraction analysis with Cu-Kα radiation, the catalyst is excellent in catalytic performance such as catalytic activity, selectivity and the like, as well as in catalyst life, and provides stable performance over a long period. Although the reason is not clear, when the crystallinity T is less than 5%, the catalyst has low selectivity for producing acrylic acid from the beginning of the reaction, whereas when the crystallinity T is more than 20%, the catalyst has low selectivity for producing acrylic acid from the beginning of the reaction and also has a short catalyst life, resulting in early degradation in performance.

The crystallinity in the present invention can be obtained by using an X-ray diffraction apparatus following the method according to JIS K-0131 (an absolute method). Thus, the crystallinity T is calculated by obtaining an X-ray diffraction intensity of all crystalline part(s) in the 20 range of 5° to 90° and a total X-ray diffraction intensity in the 20 range of 5° to 90°, and obtaining the ratio of the X-ray diffraction intensity of the all crystalline part(s) to the total X-ray diffraction intensity, as expressed by the following formula (2). Here, the total X-ray diffraction intensity means an integral intensity of the X-ray diffraction in the 20 range of 5° to 90°, and the X-ray diffraction intensity of the all crystalline part(s) means an integral intensity of the X-ray diffraction of the crystalline part(s) in the 20 range of 5° to 90°.

T=(an X-ray diffraction intensity of the all crystalline part(s))/(a total X-ray diffraction intensity)×100 (2)

The crystallinity T is obtained as follows, specifically. From a diffraction profile obtained by the X-ray diffraction analysis, a background is eliminated and then a halo pattern of an amorphous part is separated, thereby obtaining a diffraction profile of crystalline part(s). The diffraction profile of the crystalline part(s) is separated by peaks. An integral intensity (Sₙ) of the halo pattern of the amorphous part and integral intensities (S_{c1}, S_{c2}, S_{c3} . . . ) of the respective peaks of the crystalline part(s) are obtained, and the X-ray diffraction intensity S_{c} of all the crystalline part(s) is calculated from the sum of the integral intensities (S_{c1}+S_{c2}+S_{c3} . . . ) of the respective peaks of the crystalline part(s). The crystallinity T is obtained from the formula: T=S_{c}/(Sₙ+S_{c})×100.

In the catalyst of the present invention, a crystallinity ratio R, which is calculated as a ratio of a crystallinity M of the peak at 2θ=22.2±0.5° in the catalytic active component, measured by the X-ray diffraction analysis with Cu-Kα radiation, to the crystallinity T, as expressed by the following formula (1), is preferably in the range of 0.06 or more and 0.30 or less. More preferably, the crystallinity ratio is in the range of 0.07 or more and 0.25 or less.

R=M/T (1)

In the above formula (1), M represents a crystallinity of the peak at 2θ=22.2±0.5° when the catalytic active component is measured by the X-ray diffraction analysis with Cu-Kα radiation. The M can be obtained by using an X-ray diffraction apparatus following the method according to JIS K-0131 (the absolute method). Thus, the M is calculated by obtaining an X-ray diffraction intensity of a crystalline part of the peak at 2θ=22.2±0.5° and a total X-ray diffraction intensity in the 20 range of 5° to 90°, and obtaining the ratio of the X-ray diffraction intensity at 2θ=22.2±0.5° to the total X-ray diffraction intensity, as expressed by the following formula (3). Here, the X-ray diffraction intensity of the crystalline part of the peak at 2θ=22.2±0.5° means an integral intensity of the X-ray diffraction having a peak at 2θ=22.2±0.5°.

M=(an X-ray diffraction intensity of a crystalline part of the peak)/(a total X-ray diffraction intensity)×100 (3)

The crystalline part of the peak at 2θ=22.2±0.5° is speculated to be attributed to an active compound of molybdenum and vanadium, which is effective for a reaction of producing acrylic acid by gas-phase catalytic oxidation of propane and/or acrolein. The crystallinity ratio R corresponds to the ratio of the X-ray diffraction intensity of the crystalline part of the peak at 2θ=22.2±0.5° to the X-ray diffraction intensity of all the crystalline part(s) in the 20 range of 5° to 90°. Thus, by defining the crystallinity ratio R, the ratio of the specific crystalline part in the whole crystalline part is defined, whereby a catalyst for producing acrylic acid which is more excellent in catalyst performance and catalyst life and provides stable performance over a long period can be easily obtained.

The catalyst for producing acrylic acid of the present invention comprises the catalytic active component containing molybdenum and vanadium as essential elements. For the catalyst for producing acrylic acid of the present invention, it is important that the crystallinity T is within the aforementioned range, and the present invention can be applied to a catalyst which contains molybdenum and vanadium essentially as the catalytic active component. As to the catalyst which contains molybdenum and vanadium as essential elements, compositions of the catalytic active component and preparing methods are variously proposed by many companies; however, in the present invention, it is preferred that the catalyst comprises the catalytic active component having the composition expressed by the following formula (4). When the catalyst comprises the catalytic active component having the composition expressed by the following formula (4), the catalyst which is excellent in catalyst performance such as activity, an yield of acrylic acid, catalyst life and the like is easily obtained by adjusting the crystallinity T in an appropriate range:

MoₐV_{b}A_{c}B_{d}CₑD_{f}O_{z} (4)

wherein Mo is molybdenum; V is vanadium; A represents niobium and/or tungsten; B represents at least of one kind of element selected from the group consisting of chromium, manganese, iron, cobalt, nickel, copper, zinc and bismuth; C represents at least of one kind of element selected from the group consisting of tin, antimony and tellurium; D represents at least of one kind of element selected from the group consisting of titanium, aluminum, silicon and zirconium; O is oxygen; a, b, c, d, e, f and z mean atomic ratios of Mo, V, A, B, C, D and O, respectively, and meet inequalities: a=12, 1≦b≦14, 0≦c≦12, 0≦d≦10, 0≦e≦6 and 0≦f≦40, respectively; and z is a numeral value determined by oxidation states of respective elements.

The catalyst of the present invention may comprise an inert carrier for supporting the catalytic active component in addition to the catalytic component containing molybdenum and vanadium as essential elements. Examples of the inert carrier include alumina, silica, silica-alumina, titania, magnesia, steatite, cordierite, silica-magnesia, silicon carbide, silicon nitride, zeolite, and the like. A shape of the carrier is not particularly limited, and a carrier having a spherical shape, a cylindrical shape, a ring shape, or the like, may be used.

A preparing method of the catalyst of the present invention is hereinafter explained. The preparing method of a catalyst is variously proposed; and the following method may be employed, for example.

As a raw material for the catalytic active component, an oxide, a hydroxide, or a salt (e.g. an ammonium salt, a nitrate salt, a carbonate salt, a sulfate salt, an organic acid salt or the like) of each element composing the catalytic active component, an aqueous solution or a sol thereof, a compound containing a plurality of elements, or the like, is mixed with water to give an aqueous solution or an aqueous slurry (hereinafter referred to as a “mixed liquid of starting materials”), for example.

The obtained mixed liquid of starting materials is dried by a method such as heating or pressure reduction as required to obtain a catalyst precursor. For the drying method by heating, for example, a spray dryer or a drum dryer may be used, resulting in obtaining a powdery catalyst precursor. Alternatively, the mixed liquid of starting materials may be heated using a box dryer, a tunnel dryer or the like, while flowing an air, to obtain a blockish or flaky catalyst precursor. Furthermore, it is also possible to employ a method of concentrating and evaporating the mixed liquid of starting materials to dryness to give a cake-like solid matter and further heating the solid matter as described above. For the drying method by pressure reduction, for example, a vacuum dryer may be used, thereby obtaining a blockish or flaky catalyst precursor.

The obtained catalyst precursor may be gone through a pulverization step and a classification step for giving a powder having an appropriate particle size, as required, and then sent to a subsequent forming step. In some cases, the catalyst precursor may be once calcinated and then sent to the forming step. A particle size of the catalyst precursor before being sent to the forming step is not particularly limited; however, the catalyst precursor preferably has a particle size such that 90 mass % or more of the particles pass though a sieve with a mesh size of 500 μm, in view of obtaining excellent formability.

In the forming step, a compact may be obtained by forming the catalyst precursor into a specific shape by extrusion molding, tablet compression, or the like. Alternatively, a supported catalyst may be obtained by making any inert carrier having a specific shape support the mixed liquid of starting materials or the catalyst precursor as the catalytic active component (supporting method).

A shape of the compact obtained by extrusion molding, tablet compression or the like is not particularly limited, and any shape such as a spherical shape, a cylindrical shape, a ring shape, an indeterminate shape or the like, may be taken. Of course, the spherical shape does not have to be a true sphere and a substantially spherical shape will suffice. The same applies also to the cylindrical shape and the ring shape.

As the supporting method, an evaporation to dryness method in which the mixed liquid of starting materials, which is not dried and remains in the form of an aqueous solution or an aqueous slurry, is applied or attached to an inert carrier having a specific shape while heating, and then dried to make the inert carrier support the mixed liquid of starting materials, may be employed, or a granulation method in which an inert carrier is made to support a powdery catalyst precursor may be employed. Among them, as the granulation method, a centrifugal fluidizing coating method described in Japanese Unexamined Patent Application Publication No. 63-200839, or a rocking mixer method described in Japanese Unexamined Patent Application Publication No. 2004-136267, is particularly preferred. A material and shape of the inert carrier which can be used for the supporting method is explained above.

In the forming step, a forming auxiliary agent for enhancing moldability, a binder, a pore-forming agent for forming appropriate pores in a catalyst, or the like may be added. Specific examples of these agents include organic compounds such as ethylene glycol, glycerin, propionic acid, maleic acid, benzyl alcohol, propyl alcohol, butyl alcohol, phenol and the like; water; and inorganic salts such as nitric acid, ammonium nitrate, ammonium carbonate and the like.

In addition, a strengthening agent such as silica, alumina, glass fiber, silicon carbide, silicon nitride and the like can be used for the purpose of enhancing mechanical strength of the catalyst. The strengthening agent may be added to the mixed liquid of starting materials or may be mixed with the catalyst precursor.

The compact or the supported catalyst which have been obtained in the forming step is sent to a subsequent calcination step, where the compact or the supported catalyst is calcinated. The calcination temperature is preferably in the range of 350° C. or more and 450° C. or less, and more preferably in the range of 380° C. or more and 420° C. or less. The calcination period is preferably 1 hour or longer and 10 hours or shorter. A calcinating furnace used for the calcination step is not restricted, and a box furnace, a tunnel furnace or the like, which are generally used, may be used.

In the catalyst of the present invention, the crystallinity T is within the specific range, and further, the crystallinity ratio R is preferably within the specific range. For obtaining such a catalyst, it is preferred to adopt the following method. Thus, in the above preparation method of a catalyst, upon preparation of the mixed liquid of starting materials, the raw material may be fed in portions, or the feeding period of the raw material or the temperature of the mixed liquid upon mixing of the raw materials may be adjusted appropriately; or the catalyst which has been calcinated may be subjected to a hydrothermal treatment under specific conditions. Specifically, when preparing the mixed liquid of starting materials, the raw material such as molybdenum and the like may be fed in two or more portions; or when adding an aqueous solution (solution B) containing the component B in the above formula (4) to an aqueous solution (solution A) containing molybdenum and vanadium, the solution B may be added over a period of 30 seconds to 10 minutes, preferably 1 minute to 5 minutes. The temperature of the mixed liquid upon mixing the raw materials may be appropriately determined according to the raw materials to be fed. In the case that the catalyst is subjected to a hydrothermal treatment under specific conditions, the catalyst which has been calcinated may be put in an autoclave with water serving as saturated water vapor therein, and treated for 2 to 48 hours under pressure at a temperature in the range of 150° C. to 250° C., preferably 190° C. to 240° C. In the above method, it is possible to change the crystallinity ratio R by appropriately varying the composition ratio between molybdenum and vanadium in the catalytic active component.

A process for producing acrylic acid of the present invention is hereinafter explained. The process for producing acrylic acid of the present invention is a process for producing acrylic acid by gas-phase catalytic oxidation of propane and/or acrolein with molecular oxygen-containing gas, comprising the step of conducting the gas-phase catalytic oxidation in the presence of the catalyst of the present invention. According to the process for producing acrylic acid of the present invention, it is possible to stably produce acrylic acid at a high yield for a long period.

In the process for producing acrylic acid of the present invention, a reactor to be used is not restricted as long as gas-phase catalytic oxidation is conducted by using the catalyst of the present invention. Any reactor such as a fixed-bed reactor, a fluidized-bed reactor, and a moving bed reactor can be used as the reactor. Preferably, a fixed-bed reactor is used as the reactor.

As the fixed-bed reactor, a tubular reactor is preferable and a multitubular reactor is more preferable. In this case, the fixed-bed reactor comprises one or more of reaction tube(s), in which the catalyst is filled. The reaction tube is generally placed so as to be vertically-oriented in the reactor. An inner diameter of the reaction tube is not particularly limited as long as the catalyst can be filled therein; however, it is preferably in the range of 15 mm or more and 50 mm or less, more preferably 20 mm or more to 40 mm or less, and even more preferably 22 mm or more to 38 mm or less.

The catalyst to be filled in the reaction tube may be one kind or may be two or more kinds; however, the catalyst is preferably filled in the reaction tube preferably to define a plurality of reaction zones, which are provided by dividing the catalyst layer filled in the reaction tube into two or more zones in an axis direction of the reaction tube, such that the catalysts filled in the respective reaction zones have the different crystallinity ratio R from each other. Thus, it is preferable that the reaction tube has a plurality of reaction zones divided in the axis direction of the reaction tube, and the respective reaction zones are filled with the catalysts having the different crystallinity ratio R from each other. More preferably, the catalyst is filled in the reaction tube such that the crystallinity ratios R of the plurality of the reaction zones decrease in an order from an inlet to an outlet of a raw material gas containing propane and/or acrolein with molecular oxygen-containing gas. In this case, the catalysts filled in the respective reaction zones preferably have the crystallinity ratio R in the range of 0.06 or more and 0.30 or less. Further, the above catalyst filling manner may be combined with the following manner: the catalyst is filled such that the volume occupied by the catalyst decreases from the inlet to the outlet of the raw material gas; or a part of the catalyst is diluted with an inert carrier or the like.

A number of the reaction zone is determined appropriately depending on reaction conditions or a scale of the reactor. However, from an industrial viewpoint, the number of the reaction zones is preferably about 2 or more and about 6 or less, because the excessively large number of the reaction zones will cause problems such as complication of a filling operation of the catalyst.

Reaction conditions in the process for producing acrylic acid of the present invention is not particularly limited; and any reaction condition generally used in this kind of reaction can be adopted. For example, a mixed gas, composed of 1 to 15 volume % (preferably, 4 to 12 volume %) of propane and/or acrolein, 0.5 to 25 volume % (preferably, 2 to 20 volume %) of molecular oxygen, 0 to 30 volume % (preferably, 0 to 25 volume %) of steam, and inert gas such as nitrogen and the like as the residue, may be used as the raw material gas. The raw material gas may be brought into contact with the catalyst at a temperature in the range of 200° C. to 400° C., under a pressure in the range of 0.1 MPa to 1.0 MPa, at a space velocity (STP) in the range of 300 h⁻¹ to 5,000 h⁻¹. Further, as well as the mixed gas composed of propane and/or acrolein, molecular oxygen and inert gas, a mixed gas composed of acrolein obtained by a dehydration reaction of glycerin or obtained by an oxidation reaction of propylene is also available for the raw material gas. To the mixed gas, air, oxygen or the like may be added, as necessary.

### EXAMPLES

The present invention will hereinafter be described more specifically by reference to Examples; however, the present invention is not limited to these Examples. The present invention can be put into practice after appropriate modifications or variations within a range meeting the gist of the present invention, all of which are included in the technical scope of the present invention. Hereinafter, the term “part(s) by mass” may be described simply as “part(s)” for convenience sake.

Conversion rate of acrolein and an yield of acrylic acid were determined by the following equations:

Conversion rate of acrolein (mol %)=(molar quantity of reacted acrolein)/(molar quantity of fed acrolein)×100

Yield of acrylic acid (mol %)=(molar quantity of produced acrylic acid)/(molar quantity of fed acrolein)×100

An X-ray diffraction measurement was conducted using X'PertPro manufactured by Spectris Co., Ltd. A sample for the X-ray diffraction measurement was produced by passing a catalytic active component through a sieve with a mesh size of 150 μm and compression-forming about 0.5 g of the powder that passed through the sieve into tablets each having a diameter of 16 mm and a thickness of 2 mm. The sample for the X-ray diffraction measurement was used to measure the X-ray diffraction intensity with Cu-Kα radiation in the 20 range of 5° to 90° under conditions of an X-ray output of 40 mA and 45 kV, an X-ray radiation width of 15 mm, and a measurement temperature of 25° C. From the obtained X-ray diffraction profile, the crystallinity T and the crystallinity ratio R were obtained, following the method according to JIS K-0131 (an absolute method).

### (1) Experiment Example 1

### (1-1) Preparation of Catalyst

3500 parts of distilled water was stirred with the temperature maintained at 80° C., and 400 parts of ammonium paramolybdate, 116 parts of ammonium metavanadate, and 76.5 parts of ammonium paratungstate were added thereto (solution A). Separately, 400 parts of distilled water was stirred with the temperature maintained at 80° C., and 143 parts of copper nitrate was dissolved therein (solution B). While the solution A being stirred, the solution B was added to the solution A over 2 minutes to give a mixed liquid. 24.1 parts of antimony trioxide was further added to the mixed liquid, and the obtained mixture was stirred for 1 hour to give a suspension liquid. 100 parts of ammonium paramolybdate was further added to the suspension liquid, and the obtained mixture was stirred for 1 hour to give a mixed liquid of starting materials. The mixed liquid of starting materials was dried in a drum dryer to give a granular powder. The obtained granular powder was dried for about 2 hours at 210° C. under an air atmosphere. The dried granular powder was pulverized to a particle diameter of 250 μm or less to give a powdery catalyst precursor. Into a centrifugal fluidizing coating apparatus, 1500 parts of a silica-alumina spherical carrier having an average particle diameter of 4.5 mm was fed and then the catalyst precursor was fed together with a 15 mass % ammonium nitrate aqueous solution as a binder with flowing hot air of 90° C. to make the carrier support the catalyst precursor. Then, the obtained supported catalyst precursor was calcinated at 390° C. for 6 hours under an air atmosphere to give a catalyst 1. The supported ratio of the catalyst 1 was about 30 mass %, and the catalyst 1 had the following metal element composition excluding oxygen.

### Catalyst 1: Mo₁₂V_{4.2}Cu_{2.5}W_{1.2}Sb_{0.7}

The supported ratio was determined by the following equations:

Supported ratio (mass %)=(mass of a catalyst(g)−mass of a used carrier (g))/(mass of a used carrier (g))×100

In the catalyst 1, the crystallinity T in the 20 range of 5° to 90°, measured by an X-ray diffraction analysis, of the catalytic active component was 7.7%, and the crystallinity ratio R was 0.14.

### (1-2) Reaction

A reactor having a reaction tube (reaction tube length: 3,000 mm, inner diameter: 25 mm) made of stainless steel and a shell in which a heat medium is flowed and which covers the reaction tube was vertically-installed. The catalyst 1 was filled into the reactor by dropping the catalyst 1 from the top of the reaction tube so that the layer length comes to be 2900 mm. While maintaining the temperature of the heat medium (a reaction temperature) at 263° C., a mixed gas consisting of 7 volume % of acrolein, 8.5 volume % of oxygen, 10 volume % of steam and an inert gas such as nitrogen as the residue was introduced from the bottom of the reaction tube filled with the catalyst at a space velocity (STP) of 1650 hr⁻¹, thereby conducting oxidation reaction of acrolein. The results of the conversion rate of acrolein and the yield of acrylic acid are shown in Table 1. Further, the result after continuous reaction for 8000 hours, that was conducted while adjusting the reaction temperature appropriately, is also shown in Table 1. In Experiment Example 1, the initial yield of acrylic acid was as high as 95.0 mol %, and the decrease of the yield of acrylic acid after 8000 hours was suppressed to be only in the amount of 0.3 mol %. In Experiment Example 1, acrylic acid was produced stably at a high yield for a long period.

### (2) Experiment Example 2

A catalyst 2 was prepared in the same manner as in Experiment Example 1, except that the amounts of ammonium paramolybdate added to the solution A and the suspension liquid were changed to 500 parts and 0 part, respectively, and the period for adding the solution B to the solution A was changed to 1 minute in Experiment Example 1. The supported ratio of the catalyst 2 was about 30 mass %, and the metal element composition of the catalyst 2 was the same as that of the catalyst 1. In the catalyst 2, the crystallinity T was 10.5% and the crystallinity ratio R was 0.10.

The obtained catalyst 2 was filled in the same manner as in Experiment Example 1 and oxidation reaction of acrolein was conducted under the same conditions as in Experiment Example 1. The results are shown in Table 1. Also in Experiment Example 2, the initial yield of acrylic acid was as high as 95.0 mol % or more.

### (3) Experiment Example 3

A catalyst 3 was prepared in the same manner as in Experiment Example 2, except that the maintaining temperature of the solutions A and B were changed to 90° C., and the period for adding the solution B to the solution A was changed to within 10 seconds in Experiment Example 2. The supported ratio of the catalyst 3 was about 30 mass %, and the metal element composition of the catalyst 3 was the same as that of the catalyst 2. In the catalyst 3, the crystallinity T was 20.8% and the crystallinity ratio R was 0.04.

The obtained catalyst 3 was filled in the same manner as in Experiment Example 1 and oxidation reaction of acrolein was conducted under the same conditions as in Experiment Example 1. The results are shown in Table 1. Further, the result after continuous reaction for 8000 hours, that was conducted while adjusting the reaction temperature appropriately, is also shown in Table 1. In Experiment Example 3, since the crystallinity T of the catalyst 3 was more than 20%, the initial yield of acrylic acid was as low as 92.8 mol %. After 8000 hours, the yield of acrylic acid further decreased, and the amount of the decrease of the yield of acrylic acid was as much as 2.4 mol % compared to the initial stage.

### (4) Experiment Example 4

A catalyst 4 was prepared in the same manner as in Experiment Example 1, except that the maintaining temperature of the solutions A and B were changed to 70° C., the amounts of ammonium paramolybdate added to the solution A and the suspension liquid were changed to 100 parts and 400 parts, respectively, the amount of ammonium metavanadate was changed to 138 parts, and the period for adding the solution B to the solution A was changed to 12 minutes in Experiment Example 1. The supported ratio of the catalyst 4 was about 30 mass %, and the catalyst 4 had the following metal element composition excluding oxygen.

### Catalyst 4: Mo₁₂V₅Cu_{2.5}W_{1.2}Sb_{0.7}

In the catalyst 4, the crystallinity T was 5.5% and the crystallinity ratio R was 0.31.

The obtained catalyst 4 was filled in the same manner as in Experiment Example 1 and oxidation reaction of acrolein was conducted under the same conditions as in Experiment Example 1. The results are shown in Table 1. Further, the result after continuous reaction for 8000 hours, that was conducted while adjusting the reaction temperature appropriately, is also shown in Table 1. In Experiment Example 4, since the catalyst 4 had a somewhat low crystallinity T of 5.5% and a high crystallinity ratio R of 0.31, the yield of acrylic acid was lower than those in Experiment Examples 1 and 2, that is, 94.2 mol % in the initial stage and 93.7 mol % after 8000 hours.

### (5) Experiment Example 5

A catalyst 5 was prepared in the same manner as in Experiment Example 4, except that the maintaining temperature of the solutions A and B were changed to 80° C., the amounts of ammonium paramolybdate added to the solution A and the suspension liquid were changed to 0 part and 500 parts, respectively, and the period for adding the solution B to the solution A was changed to 20 minutes in Experiment Example 4. The supported ratio of the catalyst 5 was about 30 mass %, and the metal element composition of the catalyst 5 was the same as that of the catalyst 4. In the catalyst 5, the crystallinity T was 4.7% and the crystallinity ratio R was 0.35.

The obtained catalyst 5 was filled in the same manner as in Experiment Example 1 and oxidation reaction of acrolein was conducted under the same conditions as in Experiment Example 1. The results are shown in Table 1. Further, the result after continuous reaction for 8000 hours, that was conducted while adjusting the reaction temperature appropriately, is also shown in Table 1. In Experiment Example 5, since the crystallinity T of the catalyst 5 was less than 5.0%, the initial yield of acrylic acid was resulted in a low value of 92.5 mol %. After 8000 hours, the yield of acrylic acid further decreased, and the amount of the decrease of the yield of acrylic acid was as much as 1.6 mol % compared to the initial stage.

### (6) Experiment Example 6

A catalyst 6 was prepared in the same manner as in Experiment Example 1, except that the maintaining temperature of the solutions A and B were changed to 90° C., the amounts of ammonium paramolybdate added to the solution A and the suspension liquid were changed to 450 parts and 50 parts, respectively, the amount of ammonium metavanadate was changed to 82.8 parts, the amount of copper nitrate was changed to 228 parts and the solution B was added to the solution A in a oner in Experiment Example 1. The supported ratio of the catalyst 6 was about 30 mass %, and the catalyst 6 had the following metal element composition excluding oxygen.

### Catalyst 6: Mo₁₂V₃Cu₄W_{1.2}Sb_{0.7}

In the catalyst 6, the crystallinity T was 19.1% and the crystallinity ratio R was 0.05.

The obtained catalyst 6 was filled in the same manner as in Experiment Example 1 and oxidation reaction of acrolein was conducted under the same conditions as in Experiment Example 1. The results are shown in Table 1. In Experiment Example 6, since the catalyst 6 had a somewhat high crystallinity T of 19.1% and a low crystallinity ratio R of 0.05, the initial yield of acrylic acid was lower than those in Experiment Examples 1 and 2, that is, 94.2 mol %.

### (7) Experiment Example 7

A catalyst 7 was prepared in the same manner as in Experiment Example 6, except that the maintaining temperature of the solutions A and B were changed to 80° C., and the amounts of ammonium paramolybdate added to the solution A and the suspension liquid were changed to 0 part and 500 parts in Experiment Example 6. The supported ratio of the catalyst 7 was about 30 mass %, and the metal element composition of the catalyst 7 was the same as that of the catalyst 6. In the catalyst 7, the crystallinity T was 4.6% and the crystallinity ratio R was 0.05.

The obtained catalyst 7 was filled in the same manner as in Experiment Example 1 and oxidation reaction of acrolein was conducted under the same conditions as in Experiment Example 1. The results are shown in Table 1. In Experiment Example 7, since the crystallinity T of the catalyst 7 was less than 5.0%, the initial yield of acrylic acid was resulted in a low value of 92.6 mol %.

### (8) Experiment Example 8

A catalyst 8 was prepared in the same manner as in Experiment Example 1, except that the amount of ammonium metavanadate was changed to 110 parts, the amount of antimony trioxide was changed to 34.4 parts, and the period for adding the solution B to the solution A was changed to 30 seconds in Experiment Example 1. The supported ratio of the catalyst 8 was about 32 mass %, and the catalyst 8 had the following metal element composition excluding oxygen.

### Catalyst 8: Mo₁₂V₄Cu_{3.5}W_{1.2}Sb₁

In the catalyst 8, the crystallinity T was 13.2% and the crystallinity ratio R was 0.08.

The obtained catalyst 8 was filled in the same manner as in Experiment Example 1 and oxidation reaction of acrolein was conducted under the same conditions as in Experiment Example 1. The results are shown in Table 1. In Experiment Example 8, the initial yield of acrylic acid was as high as 95.0 mol % or more.

### (9) Experiment Example 9

A catalyst 9 was prepared in the same manner as in Experiment Example 1, except that the maintaining temperature of the solutions A and B were changed to 70° C., the amounts of ammonium paramolybdate added to the solution A and the suspension liquid were changed to 300 parts and 200 parts, respectively, the amounts of ammonium metavanadate, copper nitrate and antimony trioxide were changed to 138 parts, 171 parts and 34.4 parts, respectively, and the period for adding the solution B to the solution A was changed to 5 minutes in Experiment Example 1. The supported ratio of the catalyst 9 was about 32 mass %, and the catalyst 9 had the following metal element composition excluding oxygen.

### Catalyst 9: Mo₁₂V₅Cu₃W_{1.2}Sb₁

In the catalyst 9, the crystallinity T was 7.2% and the crystallinity ratio R was 0.24.

The obtained catalyst 9 was filled in the same manner as in Experiment Example 1 and oxidation reaction of acrolein was conducted under the same conditions as in Experiment Example 1. The results are shown in Table 1. In Experiment Example 9, the initial yield of acrylic acid was as high as 95.0 mol % or more.

### (10) Experiment Example 10

A catalyst 10 was prepared in the same manner as in Experiment Example 9, except that the amounts of ammonium paramolybdate added to the solution A and the suspension liquid were changed to 150 parts and 350 parts, respectively, and the period for adding the solution B to the solution A was changed to 8 minutes in Experiment Example 9. The supported ratio of the catalyst 10 was about 32 mass %, and the metal element composition of the catalyst 10 was the same as that of the catalyst 9. In the catalyst 10, the crystallinity T was 6.4% and the crystallinity ratio R was 0.29.

The obtained catalyst 10 was filled in the same manner as in Experiment Example 1 and oxidation reaction of acrolein was conducted under the same conditions as in Experiment Example 1. The results are shown in Table 1. In Experiment Example 10, the initial yield of acrylic acid was a relatively high value of 94.4 mol %.

### (11) Experiment Example 11

A catalyst 11 was prepared in the same manner as in Experiment Example 1, except that the maintaining temperature of the solutions A and B were changed to 90° C., the amounts of ammonium paramolybdate added to the solution A and the suspension liquid were changed to 350 parts and 150 parts, respectively, the amounts of ammonium metavanadate, copper nitrate and antimony trioxide were changed to 82.8 parts, 228 parts and 34.4 parts, respectively, and the period for adding the solution B to the solution A was changed to 30 seconds in Experiment Example 1. The supported ratio of the catalyst 11 was about 31 mass %, and the catalyst 11 had the following metal element composition excluding oxygen.

### Catalyst 11: Mo₁₂V₃Cu₄W_{1.2}Sb₁

In the catalyst 11, the crystallinity T was 15.9% and the crystallinity ratio R was 0.07.

The obtained catalyst 11 was filled in the same manner as in Experiment Example 1 and oxidation reaction of acrolein was conducted under the same conditions as in Experiment Example 1. The results are shown in Table 1. In Experiment Example 11, the initial yield of acrylic acid was a relatively high value of 94.8 mol %.

TABLE 1

| | | | | Passage | Reaction | Conversion Rate | Yield of |
|---|---|---|---|---|---|---|---|
| | Catalyst | Crystallinity | Crystallinity | of Time | Temperature | of Acrolein | Acrylic Acid |
| | No. | T (%) | Ratio R | (hours) | (° C.) | (mol %) | (mol %) |
| Experiment | 1 | 7.7 | 0.14 | initial | 263 | 99.6 | 95.8 |
| Example 1 | | | | 8000 | 268 | 99.5 | 95.5 |
| Experiment | 2 | 10.5 | 0.10 | initial | 263 | 99.5 | 95.6 |
| Example 2 | | | | | | | |
| Experiment | 3 | 20.8 | 0.04 | initial | 263 | 98.8 | 92.8 |
| Example 3 | | | | 8000 | 279 | 97.3 | 90.4 |
| Experiment | 4 | 5.5 | 0.31 | initial | 263 | 98.9 | 94.2 |
| Example 4 | | | | 8000 | 271 | 98.6 | 93.7 |
| Experiment | 5 | 4.7 | 0.35 | initial | 263 | 98.7 | 92.5 |
| Example 5 | | | | 8000 | 276 | 97.6 | 90.9 |
| Experiment | 6 | 19.1 | 0.05 | initial | 263 | 99.1 | 94.2 |
| Example 6 | | | | | | | |
| Experiment | 7 | 4.6 | 0.05 | initial | 263 | 98.7 | 92.6 |
| Example 7 | | | | | | | |
| Experiment | 8 | 13.2 | 0.08 | initial | 263 | 99.4 | 95.2 |
| Example 8 | | | | | | | |
| Experiment | 9 | 7.2 | 0.24 | initial | 263 | 99.5 | 95.6 |
| Example 9 | | | | | | | |
| Experiment | 10 | 6.4 | 0.29 | initial | 263 | 99.0 | 94.4 |
| Example 10 | | | | | | | |
| Experiment | 11 | 15.9 | 0.07 | initial | 263 | 99.3 | 94.8 |
| Example 11 | | | | | | | |

### (12) Experiment Example 12

### (12-1) Preparation of Catalyst

A catalyst 12 was prepared in the same manner as in Experiment Example 1, except that the carrier used in Experiment Example 1 was changed to a silica-alumina spherical carrier having an average particle diameter of 7.5 mm. The supported ratio of the catalyst 12 was about 30 mass %, and the metal element composition of the catalyst 12 was the same as that of the catalyst 1. The crystallinity T and the crystallinity ratio R of the catalyst 12 were the same as those of the catalyst 1.

### (12-2) Reaction

A reactor having a reaction tube (reaction tube length: 3,000 mm, inner diameter: 25 mm) made of stainless steel and a shell in which a heat medium is flowed and which covers the reaction tube was vertically-installed. The catalysts 12 and 2 were filled into the reactor by sequentially dropping the catalyst 12 and the catalyst 2 from the top of the reaction tube so that the layer lengths come to be 900 mm for the catalyst 12 and 2000 mm for the catalyst 2.

While maintaining the temperature of the heat medium (a reaction temperature) at 263° C., a mixed gas consisting of 8.5 volume % of acrolein, 10.2 volume % of oxygen, 8 volume % of steam and an inert gas such as nitrogen as the residue was introduced from the bottom of the reaction tube filled with the catalysts at a space velocity (STP) of 1700 hr⁻¹, thereby conducting oxidation reaction of acrolein. In Experiment Example 12, the catalyst 12 was placed on an inlet side, with respect to the flow of the mixed gas, of the reaction tube, and the catalyst 2 was placed on an outlet side, with respect to the flow of the mixed gas, of the reaction tube. The reaction was conducted continuously for 8000 hours while adjusting the reaction temperature appropriately. The results are shown in Table 2. In Experiment Example 12, two kinds of catalysts, i.e. the catalysts 2 and 12, each of which has the crystallinity ratio R in the range of 0.06 or more and 0.30 or less, were filled into the reaction tube. The catalyst 12 having the higher crystallinity ratio R was placed on the inlet side, and the catalyst 2 having the lower crystallinity ratio R was placed on the outlet side. As a result, the yield of acrylic acid was a high value of 95 mol % or more both in the initial stage and after 8000 hours.

### (13) Experiment Example 13

A catalyst 13 was prepared in the same manner as in Experiment Example 2, except that the carrier used in Experiment Example 2 was changed to a silica-alumina spherical carrier having an average particle diameter of 7.5 mm. The supported ratio of the catalyst 13 was about 30 mass %, and the metal element composition of the catalyst 13 was the same as that of the catalyst 2. The crystallinity T and the crystallinity ratio R of the catalyst 13 were the same as those of the catalyst 2.

Into the same reaction tube as in the Experiment Example 12, the catalysts 13 and 1 were filled by sequentially dropping the catalyst 13 and the catalyst 1 from the top of the reaction tube so that the layer lengths come to be 900 mm for the catalyst 13 and 2000 mm for the catalyst 1, and oxidation reaction of acrolein was conducted under the same conditions as in Experiment Example 12. In Experiment Example 13, the catalyst 13 was placed on the inlet side, with respect to the flow of the mixed gas, of the reaction tube, and the catalyst 1 was placed on the outlet side, with respect to the flow of the mixed gas, of the reaction tube. The results are shown in Table 2. In Experiment Example 13, two kinds of catalysts, i.e. the catalysts 1 and 13, each of which has the crystallinity ratio R in the range of 0.06 or more and 0.30 or less, were filled into the reaction tube. The catalyst 13 having the lower crystallinity ratio R was placed on the inlet side, and the catalyst 1 having the higher crystallinity ratio R was placed on the outlet side. Also in Experiment Example 13, the yield of acrylic acid was maintained to be high both in the initial stage and after 8000 hours; however, the performance in Experiment Example 13 was lower than that in Experiment Example 12.

### (14) Experiment Example 14

A catalyst 14 was prepared in the same manner as in Experiment Example 3, except that the carrier used in Experiment Example 3 was changed to a silica-alumina spherical carrier having an average particle diameter of 7.5 mm. The supported ratio of the catalyst 14 was about 30 mass %, and the metal element composition of the catalyst 14 was the same as that of the catalyst 3. The crystallinity T and the crystallinity ratio R of the catalyst 14 were the same as those of the catalyst 3.

Into the same reaction tube as in the Experiment Example 12, the catalysts 14 and 3 were filled by sequentially dropping the catalyst 14 and the catalyst 3 from the top of the reaction tube so that the layer lengths come to be 900 mm for the catalyst 14 and 2000 mm for the catalyst 3, and oxidation reaction of acrolein was conducted under the same conditions as in Experiment Example 12. In Experiment Example 14, the catalyst 14 was placed on the inlet side, with respect to the flow of the mixed gas, of the reaction tube, and the catalyst 3 was placed on the outlet side, with respect to the flow of the mixed gas, of the reaction tube. The results are shown in Table 2. In Experiment Example 14, the catalysts 3 and 14, which have the same crystallinity ratio R each other out of the range of 0.06 to 0.30, were filled into the reaction tube. The yield of acrylic acid in Experiment Example 14 was lower than those in the Experiment Examples 12 and 13.

### (15) Experiment Example 15

A catalyst 15 was prepared in the same manner as in Experiment Example 5, except that the carrier used in Experiment Example 5 was changed to a silica-alumina spherical carrier having an average particle diameter of 7.5 mm. The supported ratio of the catalyst 15 was about 30 mass %, and the metal element composition of the catalyst 15 was the same as that of the catalyst 5. The crystallinity T and the crystallinity ratio R of the catalyst 15 were the same as those of the catalyst 5.

Into the same reaction tube as in the Experiment Example 12, the catalysts 15 and 3 were filled by sequentially dropping the catalyst 15 and the catalyst 3 from the top of the reaction tube so that the layer lengths come to be 900 mm for the catalyst 15 and 2000 mm for the catalyst 3, and oxidation reaction of acrolein was conducted under the same conditions as in Experiment Example 12. In Experiment Example 15, the catalyst 15 was placed on the inlet side, with respect to the flow of the mixed gas, of the reaction tube, and the catalyst 3 was placed on the outlet side, with respect to the flow of the mixed gas, of the reaction tube. The results are shown in Table 2. In Experiment Example 15, two kinds of catalysts, i.e. the catalysts 3 and 15, each of which has the crystallinity ratio R out of the range of 0.06 to 0.30, were filled into the reaction tube. The catalyst 15 having the higher crystallinity ratio R was placed on the inlet side, and the catalyst 3 having the lower crystallinity ratio R was placed on the outlet side. The yield of acrylic acid in Experiment Example 15 was lower than those in the Experiment Examples 12 and 13.

TABLE 2

| | | Passage | Reaction | Conversion Rate | Yield of |
|---|---|---|---|---|---|
| | | of Time | Temperature | of Acrolein | Acrylic Acid |
| | | (hours) | (° C.) | (mol %) | (mol %) |
| Experiment | inlet side: catalyst 12 (R = 0.14) | initial | 263 | 99.2 | 95.3 |
| Example 12 | outlet side: catalyst 2 (R = 0.10) | 8000 | 268 | 99.2 | 95.1 |
| Experiment | inlet side: catalyst 13 (R = 0.10) | initial | 263 | 99.1 | 95.0 |
| Example 13 | outlet side: catalyst 1 (R = 0.14) | 8000 | 271 | 99.0 | 94.7 |
| Experiment | inlet side: catalyst 14 (R = 0.04) | initial | 263 | 98.5 | 92.2 |
| Example 14 | outlet side: catalyst 3 (R = 0.04) | 8000 | 278 | 97.5 | 90.8 |
| Experiment | inlet side: catalyst 15 (R = 0.35) | initial | 263 | 98.6 | 92.3 |
| Example 15 | outlet side: catalyst 3 (R = 0.04) | 8000 | 276 | 97.6 | 91.0 |

1. A catalyst for producing acrylic acid from propane and/or acrolein by gas-phase catalytic oxidation with molecular oxygen-containing gas, comprising
a catalytic active component containing molybdenum and vanadium as essential elements, and having a crystallinity T of 5% or more and 20% or less in the 20 range of 5° to 90°, measured by an X-ray diffraction analysis with Cu-Kα radiation.

2. The catalyst for producing acrylic acid according to claim 1, wherein a crystallinity ratio R expressed by the following formula (1) is in the range of 0.06 or more and 0.30 or less.
R=M/T (1)
(M represents a crystallinity of the peak at 2θ=22.2±0.5° in the catalytic active component, measured by the X-ray diffraction analysis with Cu-Kα radiation.)

3. The catalyst for producing acrylic acid according to claim 1, further comprising an inert carrier for supporting the catalytic active component.

4. A process for producing acrylic acid by gas-phase catalytic oxidation of propane and/or acrolein with molecular oxygen, comprising the step of conducting the gas-phase catalytic oxidation in the presence of the catalyst according to claim 1.

5. The process for producing acrylic acid according to claim 4, wherein the catalyst is filled in a reaction tube of a fixed-bed reactor.

6. The process for producing acrylic acid according to claim 5, wherein
the reaction tube has a plurality of reaction zones divided in an axis direction of the reaction tube, and
the respective reaction zones are filled with the catalysts having the different crystallinity ratio R from each other.

7. The process for producing acrylic acid according to claim 6, wherein
the crystallinity ratios R of the plurality of the reaction zones decreases in an order from an inlet to an outlet of a raw material gas containing propane and/or acrolein with molecular oxygen-containing gas.
